(19) United States
(12) Patent Application Publication
(10) Pub. No.: US 2025/0332374 A1
(43) Pub. Date: Oct. 30, 2025

(54) Health Intervention and Correction Methods, Systems, and Devices Based on Vision and Sensors

(71) Applicant: AIH LLC, Houston, TX (US)

(72) Inventors: Yan LI, Houston, TX (US); Hanwen GU, Houston, TX (US); Yizuo CHEN, Houston, TX (US)

(21) Appl. No.: 18/650,466

(22) Filed: Apr. 30, 2024

(51) Int. Cl. A61M 21/02; A61H 1/02; A61H 9/00; A61M 21/00; G06T 7/00; G06T 7/246

(52) U.S. Cl. CPC A61M 21/02; A61H 1/0292; A61H 9/0078; G06T 7/0014; G06T 7/248; A61H 2203/0443; A61H 2230/625; A61M 2021/0022; G06T 2207/30196; G06T 2207/30232

(57) ABSTRACT

The present disclosure provides a health intervention and correction method, system, apparatus, and device based on vision and sensors. The method includes: acquiring various motion monitoring data obtained by detecting a user in a target state; determining a target motion model of the user based on the various motion monitoring data; wherein the target motion model is used to indicate the state information of the user at each detection moment; comparing the target motion model with a target reference motion model to obtain comparison results; and generating health intervention and correction information based on the comparison results, and sending the health intervention and correction information to a health intervention device.

### FIELD OF THE INVENTION

The present disclosure relates to the field of health management technologies, and in particular to a health intervention and correction method, system, and device based on vision and sensors.

### BACKGROUND OF THE INVENTION

In today's digital age, sedentary lifestyles, extended working hours, and the increasing prevalence of electronic devices have led to a rise in health issues related to the spine, joints, musculoskeletal and nervous systems. These prevalent health problems pose significant challenges to various demographics, such as office workers, athletes, the elderly, and people with physical disabilities.

In the related art, related issues are solved through manual assistance, physical exercise, and machine assistance. However, these solutions provided by the above technologies do not meet everyone's unique health needs and fail to adapt over time to their progress, resulting in poorly targeted health intervention plans, which often hinder the effectiveness of current health intervention measures.

### SUMMARY OF THE INVENTION

The present disclosure provides at least one health intervention and correction method, system, device, and equipment based on vision and sensors.

According to a first aspect of the present disclosure, embodiments of the present disclosure provide a health intervention and correction method based on vision and sensors, including:
acquiring various motion monitoring data obtained by detecting a user in a target state;
determining a target motion model of the user based on the various motion monitoring data, wherein the target motion model is used to indicate state information of the user at each detection moment;
comparing the target motion model with a target reference model to obtain comparison results; and
generating health intervention and correction information based on the comparison results, and sending the health intervention and correction information to a health intervention device.

In an optional embodiment, acquiring the various motion monitoring data obtained by detecting the user in the target state includes:
acquiring motion monitoring data obtained by performing motion detection on the user by a plurality of wearable devices in communication connection with a terminal device to obtain the various motion monitoring data.

In an optional embodiment, the various motion monitoring data is obtained by performing motion detection on various monitoring parts of the user, wherein the monitoring parts include but are not limited to the following parts: skeletons, joints and muscles; the motion monitoring data include but are not limited to the following types: visual images, photoelectric data, neuroelectrophysiological data, audio data and speed data.

In an optional embodiment, determining the target motion model of the user based on the various motion monitoring data includes:
transforming each of the various motion monitoring data into motion posture data of the user; and
performing data fusion on the motion posture data obtained after transforming the various motion monitoring data based on the detection moment of the motion posture data to obtain the target motion model.

In an optional embodiment, performing data fusion on the motion posture data obtained after transforming the various motion monitoring data based on the detection moment of the motion posture data to obtain the target motion model includes:
determining motion posture data with the same detection moment in the motion posture data obtained after transforming the various motion monitoring data;
processing the motion posture data with the same detection moment to obtain target motion posture data at the detection moment; and
determining the target motion model based on the target motion posture data at each detection moment.

In an optional embodiment, the target reference model is a reference motion model, and comparing the target motion model with the target reference model to obtain the comparison results includes:
determining object information of the user based on the target motion model, wherein the object information is used to indicate a motion state of the user and/or object attributes;
determining a reference motion model matched with the object information, wherein the matched reference motion model is used to indicate expected posture information of the user under the motion state; and
comparing the matched reference motion model with the target motion model to obtain the comparison results.

In an optional embodiment, comparing the target motion model with the target reference model to obtain the comparison results includes:
determining target data pairs with corresponding timestamps in the target motion model and the target reference model;
determining a data similarity of the target data pairs; and
determining the comparison results based on the data similarity of each of the target data pairs.

In an optional embodiment, generating health intervention and correction information based on the comparison results, and sending the health intervention and correction information to the health intervention device includes:
determining a model difference between the target motion model and the target reference model based on the comparison results;
determining a health intervention strategy matched with the model difference;
determining a health intervention device matched with the health intervention strategy and generating the health intervention and correction information matched with the health intervention strategy; and
sending the health intervention and correction information to the matched health intervention device.

In an optional embodiment, generating health intervention and correction information based on the comparison results, and sending the health intervention and correction information to the health intervention device includes:
determining a model difference between the target motion model and the target reference model based on the comparison results;
determining a health intervention strategy matched with the model difference;
determining a health intervention device matched with the health intervention strategy and generating the health intervention and correction information matched with the health intervention strategy; and
sending the health intervention and correction information to the matched health intervention device.

In an optional embodiment, the target reference model is medical image data, and comparing the target motion model with the target reference model to obtain comparison results includes:
extracting state information of monitoring parts of the user based on the target motion model, wherein the monitoring parts include but are not limited to the following parts: skeletons, joints and muscular systems; and
comparing the state information of the target monitoring parts with an expected skeletal state of the target monitoring parts in the medical image data to obtain the comparison results.

In an optional embodiment, the target motion model is a sleep posture model determined based on body state data obtained from the user in a sleep state, and the target reference model is a sleep reference model;
acquiring various motion monitoring data obtained by detecting a user in a target state includes:
acquiring various body state data obtained by performing posture measurement on the user in a sleep state through sensors of various dimensions, wherein the body state data includes at least one of the following types of data: sleep posture monitoring data, body photoelectric monitoring signals, body myoelectric monitoring signals and body vital sign signals; and
comparing the target motion model with the target reference model to obtain the comparison results includes:
comparing the sleep posture model with the sleep reference model to obtain the comparison results, wherein the sleep reference model is used to indicate an expected sleep posture of the user.

In an optional embodiment, generating health intervention and correction information based on the comparison results, and sending the health intervention and correction information to the health intervention device includes:
in response to determining that a posture difference between the sleep posture of the user and the expected sleep posture is large based on the comparison results, sending the health intervention and correction information to a sleep intervention device of the user, wherein the health intervention and correction information is used to adjust sleep posture of the user through the sleep intervention device.

In an optional embodiment, generating health intervention and correction information based on the comparison results includes:
generating health intervention and correction information matched with the health intervention device based on the comparison results, wherein the health intervention and correction information includes the following types: vibration information, video information, audio information, textual information and command information.

According to a second aspect of the present disclosure, there is provided a health intervention and correction system based on vision and sensors, including multiple sensors and a processor; wherein
the multiple sensors are configured to perform motion detection on a user in a target state to obtain various motion monitoring data; and
the processor is configured to acquire the various motion monitoring data and determine a target motion model of the user based on the various motion monitoring data, wherein the target motion model is used to indicate state information of the user at each detection moment; compare the target motion model with a target reference model to obtain comparison results; and generate health intervention and correction information based on the comparison results, and send the health intervention and correction information to a health intervention device.

In an optional embodiment, the processor includes a processing module, a comparison module, and an intervention module;
the processing module is configured to acquire the various motion monitoring data and determine the target motion model of the user based on the various motion monitoring data;
the comparison module is configured to compare the target motion model with the target reference model to obtain the comparison results; and
the intervention module is configured to generate health intervention and correction information based on the comparison results and send the health intervention and correction information to the health intervention device.

In an optional embodiment, the multiple sensors include wearable devices and camera devices, wherein the wearable devices and the camera devices are configured to connect to the processor via wireless or wired connections.

According to a third aspect of the present disclosure, there is also provided an electronic device, including: a processor, a memory, and a bus; wherein the memory stores machine-readable instructions executable by the processor; when the electronic device is operational, the processor communicates with the memory via the bus, and when the machine-readable instructions are executed by the processor, they carry out the steps of the first aspect, or any possible embodiment of the first aspect.

According to a fourth aspect of the present disclosure, there is also provided a computer-readable storage medium storing a computer program, wherein the computer program, when executed by a processor, causing the processor to carry out the steps of the first aspect, or any possible embodiment of the first aspect.

In the embodiments of the present disclosure, initially, various motion monitoring data obtained by detecting the user in a target state are acquired; then, the target motion model of the user is determined based on the various motion monitoring data, state information of the user at each detection moment can be determined through the target motion model; subsequently, health intervention and correction information of the user can be generated by comparing the target motion model with a target reference model, and the health intervention and correction information is sent to the health intervention device.

In the above-mentioned embodiments, by determining the target motion model based on the various motion monitoring data of the user, a personalized health model can be generated for the user; by comparing the target motion model with the target reference model to generate health intervention and correction information, more accurate health intervention and correction information can be generated for the user, thereby providing more precise intervention measures for the user.

To make the purposes, characteristics, and advantages of the present disclosure more evident and understandable, the following are preferable embodiments explained in detail in conjunction with the accompanying drawings.

### BRIEF DESCRIPTION OF DRAWINGS

To clarify the technical solutions of the embodiments of the present disclosure more clearly, the drawings used in the embodiments are briefly introduced below. These drawings are incorporated into the description and constitute a part of the present disclosure, illustrating embodiments in accordance with the present disclosure, and are used, together with the description, to explain the principles of the present disclosure. It should be understood that the following drawings illustrate only some embodiments of the present disclosure and are therefore not to be considered limiting of its scope. For those skilled in the art, other related drawings can be obtained from these figures without creative effort.

FIG. 1 illustrates a flowchart of a health intervention and correction method based on vision and sensors provided by the embodiments of the present disclosure.

FIG. 2 illustrates a flowchart of a specific method for determining a target motion model of a user based on various motion monitoring data in a health intervention and correction method based on vision and sensors provided by the embodiments of the present disclosure.

FIG. 3 illustrates a flowchart of a specific method for comparing a target motion model with a target reference model to obtain comparison results in a health intervention and correction method based on vision and sensors provided by the embodiments of the present disclosure.

FIG. 4 illustrates a flowchart of another specific method for comparing a target motion model with a target reference model to obtain comparison results in a health intervention and correction method based on vision and sensors provided by the embodiments of the present disclosure.

FIG. 5A illustrates a schematic diagram of a setting position of a health intervention device provided by the embodiments of the present disclosure.

FIG. 5B illustrates a schematic diagram of a setting position of another health intervention device provided by the embodiments of the present disclosure.

FIG. 6 illustrates a schematic diagram of a health intervention and correction system based on vision and sensors provided by the embodiments of the present disclosure.

FIG. 7 illustrates a schematic diagram of an electronic device provided by the embodiments of the present disclosure.

### DETAILED DESCRIPTION OF THE INVENTION

To make the objectives, technical solutions, and advantages of these embodiments of the present disclosure clearer, the technical solutions in the embodiments of the present disclosure are described clearly and completely in conjunction with the drawings in the embodiments of the present disclosure. Obviously, the described embodiments are only a part of the embodiments of the present disclosure, not all of them. The components presented in the drawings of the present disclosure can be arranged and designed in a variety of different configurations. Therefore, the detailed description of the embodiments of the present disclosure provided in the drawings is not intended to limit the scope of the claimed disclosure but merely represents selected embodiments. All other embodiments obtained by a person skilled in the art from the embodiments of the present disclosure without creative work are within the scope of protection of the present disclosure.

It should be noted that similar reference numbers and letters in the drawings below represent similar terms. Therefore, once a term is defined in one drawing, it does not need to be further defined and explained in subsequent drawings.

The term “and/or” used herein is merely for describing an associated relationship that indicates that there can be three relationships. For example, A and/or B can indicate: A exists alone, both A and B exist together, or B exists alone. Additionally, the term “at least one” used herein indicates any one or any combination of more than one of the multiple types, e.g., including at least one of A, B, C, can indicate including any one or more elements selected from the collection composed of A, B, and C.

It has been discovered that in today's digital age, sedentary lifestyles, extended working hours, and the increasing prevalence of electronic devices have led to a rise in health issues related to the spine, joints, musculoskeletal and nervous systems. These prevalent health problems pose significant challenges to various demographics, such as office workers, athletes, the elderly, and people with physical disabilities.

In the related art, related issues are solved through manual assistance, physical exercise, and machine assistance. However, these solutions provided by the above technologies do not meet everyone's unique health needs and fail to adapt over time to their progress, resulting in poorly targeted health intervention plans, which often hinder the effectiveness of current health intervention measures.

In recent years, with technological advancements, wearable devices, smartphone applications, and camera-based systems aimed at promoting spinal and joint health have emerged. However, these tools often operate independently and are unable to provide a holistic, synchronized, and personalized health intervention and correction method based on vision and sensors.

Based on the above research, the present disclosure provides a health intervention and correction method, system, electronic device, and storage medium based on vision and sensors. First, various motion monitoring data obtained by detecting the user in a target state are acquired; then, the target motion model of the user is determined based on the various motion monitoring data, state information of the user at each detection moment can be determined through the target motion model; subsequently, health intervention and correction information of the user can be generated by comparing the target motion model with a target reference model, and the health intervention and correction information is sent to the health intervention device.

In the above-mentioned embodiments, by determining the target motion model based on the various motion monitoring data of the user, a personalized health model can be generated for the user; by comparing the target motion model with the target reference model to generate health intervention and correction information, more accurate health intervention and correction information can be generated for the user, thereby providing more precise intervention measures for the user.

For ease of understanding this embodiment, a detailed introduction to the health intervention and correction method based on vision and sensors disclosed in this embodiment is first provided. The execution body of the health intervention and correction method based on vision and sensors provided by the present disclosure is generally an electronic device with certain computing capabilities, which may include: terminal devices, servers, or other processing devices. The terminal devices may be user equipment (UE), mobile devices, user terminals, terminals, cellular phones, cordless phones, personal digital assistants (PDA), handheld devices, computing devices, vehicular devices, wearable devices, etc. In some possible embodiments, the health intervention and correction method based on vision and sensors can be implemented by a processor calling computer-readable instructions stored in a memory.

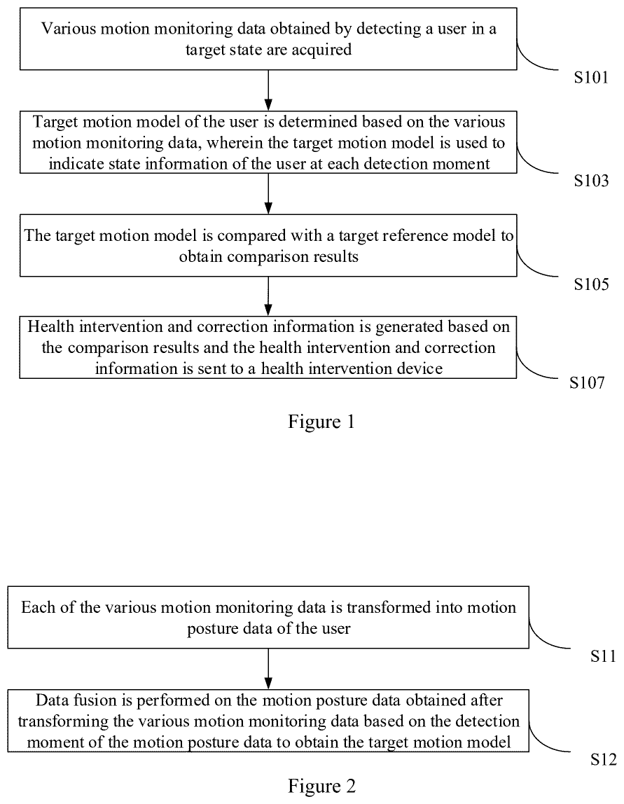

Referring to FIG. 1, FIG. 1 illustrates a flowchart of a health intervention and correction method based on vision and sensors provided by the embodiment of the present disclosure. The method includes following steps S101 to S107.

S101, various motion monitoring data obtained by detecting a user in a target state are acquired.

Here, the target state includes but is not limited to the following states: a motion state, a stationary state, and a sleep state. The various motion monitoring data can be motion monitoring data obtained by performing motion detection on the user through various sensors. The data types of the various motion monitoring data are not entirely the same.

Here, the various sensors include wearable devices and camera devices.

Here, the motion monitoring data is obtained by performing motion detection on various monitoring parts of the user, where the monitoring parts include but are not limited to the following parts: skeletons, joints, and muscles; data types of the various motion monitoring data include but are not limited to the following types: visual images, photoelectric data, neuroelectrophysiological data, audio data, and speed data.

Through the motion monitoring data, the following related motion data of the user can be determined: a motion posture of the user, a motion duration, a motion speed, a motion mode (for example, cycling or walking), a body state during a motion (such as a heart rate, a blood oxygen concentration, a blood pressure, and a pulse), a motion trajectory, and other data associated with the motion.

In the embodiments of the present disclosure, a target application can be installed in the electronic device. Here, the electronic device can obtain the motion monitoring data collected by the various sensors and send this motion monitoring data to the target application. After obtaining the motion monitoring data, the target application can perform the following steps S103 to S107. Here, if the sensor is a wearable device, a client that communicates and connects with the wearable device can be installed in the electronic device. The client can obtain the motion monitoring data collected by the wearable device through the communication connection and send this motion monitoring data to the target application.

In addition, communication connections between the electronic device with the various sensors can be established, for example, a communication connection between the electronic device and the wearable device can be established. Then, the wearable device can transmit the motion monitoring data to the electronic device through the communication connection, and after obtaining the motion monitoring data, the following steps S103 to S107 can be performed.

S103, a target motion model of the user is determined based on the various motion monitoring data, wherein the target motion model is used to indicate state information of the user at each detection moment.

Here, after obtaining the various motion monitoring data, the various motion monitoring data can be fused to obtain the target motion model.

Here, the number of the target motion model can be multiple, and the data types included in different target motion models are different. That is, multiple different target motion models in the same time period can be generated using various types of motion monitoring data.

Here, the target motion model can be represented by one or more of the following methods: a mathematical model, a statistical model, a biomechanical model, a kinematic model, or a machine learning model.

Here, the target motion model can be transformed into a motion sequence; wherein the motion sequence can indicate the state information of the user at each detection moment. Here, the detection moment is a moment when the sensor performs motion detection on the user.

For example, the motion sequence can be represented as: <(35, 24, 67), (16, 47, 24), (−24, 95, −3), (−61, 35, 24), (56, 33, 5)>; wherein each set of data in the motion sequence can be used to indicate Euler angles of the user on the X, Y, Z axes.

Alternatively, the target motion model can be a series of images; wherein the series of images contain motion images obtained by detecting the user at each detection moment.

S105, the target motion model is compared with a target reference model to obtain comparison results.

Here, the target reference model can be a reference model generated through normal motion monitoring data of the user, or a reference model generated through clinical standards. The expected posture information of the subject can be determined under normal circumstances through the target reference model.

Here, the target reference model can be represented by one or more of the following methods: a mathematical model, a statistical model, a biomechanical model, a kinematic model, or a machine learning model.

Here, the target reference model can be converted into a corresponding reference sequence, for example, the reference sequence can be represented as: <(30, 42, 70), (12, 60, 2), (−24, 95, −3), (−61, 35, 24), (56, 33, 5)>; wherein each set of data in the reference sequence is used to represent the expected posture information of the user at each detection moment under normal circumstances.

In addition, the target reference model can also be a series of images; wherein motion images included in the series of images can indicate the expected posture information of the user at each detection moment under normal circumstances.

S107, health intervention and correction information is generated based on the comparison results and the health intervention and correction information is sent to a health intervention device.

Here, after generating the health intervention and correction information, the health intervention and correction information can be sent directly to the health intervention device; or the health intervention and correction information can also be sent to the client, where the user can control the corresponding health intervention device according to the health intervention and correction information.

Here, after generating the health intervention and correction information, the health intervention and correction information can also be sent to a body manager of the user. The body manager logs into the client in the capacity of a private doctor of the user. After obtaining the health intervention and correction information, the body manager can formulate a corresponding rehabilitation strategy for the user and send this rehabilitation strategy to the user through the client. After obtaining the rehabilitation strategy, the user can control the corresponding health intervention device according to the rehabilitation strategy. Alternatively, after obtaining the health intervention and correction information, the body manager can formulate a corresponding rehabilitation strategy for the user and send this rehabilitation strategy to the health intervention device.

In the embodiments of the present disclosure, first, various motion monitoring data obtained by detecting the user in a target state are acquired; then, the target motion model of the user is determined based on the various motion monitoring data, state information of the user at each detection moment can be determined through the target motion model; subsequently, health intervention and correction information of the user can be generated by comparing the target motion model with a target reference model, and the health intervention and correction information is sent to the health intervention device.

In the embodiments of the present disclosure, the motion monitoring data is the data obtained by performing the detection after being authorized by the user. That is, before the detection, an authorization request can be sent to the electronic device and/or the wearable device, and the various motion monitoring data can be detected after detecting authorization approval information sent by the user.

In the above-mentioned embodiments, by determining the target motion model based on the various motion monitoring data of the user, a personalized health model can be generated for the user; by comparing the target motion model with the target reference model to generate health intervention and correction information, more accurate health intervention and correction information can be generated for the user, thereby providing more precise intervention measures for the user.

The steps mentioned above are described in detail in conjunction with specific embodiments.

In the embodiments of the present disclosure, first, acquiring various motion monitoring data obtained by performing motion detection on the user specifically includes the following steps:
acquiring motion monitoring data obtained by performing motion detection on the user by a plurality of wearable devices in communication connection with a terminal device to obtain the various motion monitoring data.

In the embodiments of the present disclosure, if the electronic device is a terminal device, and various wearable devices can communicate with the terminal device, then the terminal device can acquire motion monitoring data from the connected wearable devices that have performed motion detection on the user.

The wearable devices include but are not limited to the following devices: smartphones, smart watches, smart bracelets, smart earrings, smart earplugs, smart glasses, and other wearable devices.

The motion monitoring data collected by each wearable device includes a corresponding timestamp, which is used to indicate the detection moment of the corresponding motion monitoring data.

Here, the data types of motion monitoring data can be various, for example, image data, numerical data, and video data.

In the aforementioned embodiments, by acquiring motion monitoring data detected by various wearable devices, motion monitoring data of the user can be obtained more comprehensively, which can compensate for the missing data in the motion monitoring data collected by a single sensor, thereby obtaining a more accurate target motion model.

In the embodiments of the present disclosure, after acquiring the various motion monitoring data, the target motion model of the user can be determined based on the various motion monitoring data.

Here, the motion monitoring data captured by sensors can be analyzed and processed by artificial intelligence algorithms, thereby generating a personalized motion health model, that is, the target motion model.

In an optional embodiment, as shown in FIG. 2, it specifically includes the following steps:
step S11: each of the various motion monitoring data is transformed into motion posture data of the user; and
step S12: data fusion is performed on the motion posture data obtained after transforming the various motion monitoring data based on the detection moment of the motion posture data to obtain the target motion model.

In the embodiments of the present disclosure, the various motion monitoring data can be motion monitoring data of different data types, for example, the motion monitoring data can be image data, coordinate data, or textual data. At this time, each motion monitoring data can be transformed into motion posture data that indicates the posture of the user. For example, the motion posture data can be Euler angles of the user on the X, Y, Z axes at each detection moment.

After obtaining the motion posture data at each detection moment, the motion posture data can be fused based on the detection moment to obtain a fused motion posture data sequence, and this fused motion posture data sequence can be determined as the target motion model.

Here, the category of the motion posture data can be determined based on the data category in the target reference model. For example, if the data category in the target reference model is coordinate data, each motion monitoring data can be transformed into coordinate class motion posture data of the user.

Specifically, the motion posture data of the target posture object can be sorted according to the order of the detection moment, then the motion posture data corresponding to the same detection moment in the sorting results can be fused to obtain the target motion model.

Through the above processing method, it is possible to determine a personalized motion model for the user by combining multidimensional data, which can more accurately reflect motion state of the user and, therefore, generate more accurate health intervention and correction information for the user.

In an optional embodiment, the step of performing data fusion on the motion posture data obtained after transforming the various motion monitoring data to obtain the target motion model includes the following steps:
step S121, motion posture data with the same detection moment is determined in the motion posture data obtained after transforming the various motion monitoring data;
step S122, the motion posture data with the same detection moment is processed to obtain target motion posture data at the detection moment; and
step S123, the target motion model is determined based on the target motion posture data at each detection moment.

In the embodiments of the present disclosure, motion posture data of the target object can be sorted according to the order of the detection moment, and then, the motion posture data corresponding to the same detection moment in the sorting result can be determined. Next, based on the quantity of motion posture data at the same detection moment, the motion posture data can be processed.

If the number of motion posture data at the same detection moment is one, then this motion posture data is exactly the final motion posture data for that detection moment.

If there are multiple motion posture data at the same detection moment, then these multiple motion posture data can be fused to obtain a fused motion posture data for that detection moment, and this fused motion posture data can be considered as the final motion posture data for that detection moment.

For example, multiple motion posture data can be averaged to obtain the fused motion posture data; or, the reliability of each motion posture data can be evaluated, and the motion posture data with the highest reliability can be taken as the final motion posture data, where the reliability of each motion posture data can be determined based on the reliability of the each sensor that is used to collect each motion posture data.

After determining the final motion posture data for each detection moment, the target motion model can be determined based on this final motion posture data.

For a detection moment of missing the motion posture data, it is also possible to estimate the missing motion posture data for that detection moment based on the motion posture data from other detection moments before and/or after that detection moment.

Through the above processing method, the motion state of the user can be more accurately reflected, and then more accurate and reasonable health intervention and correction information can be generated for the user.

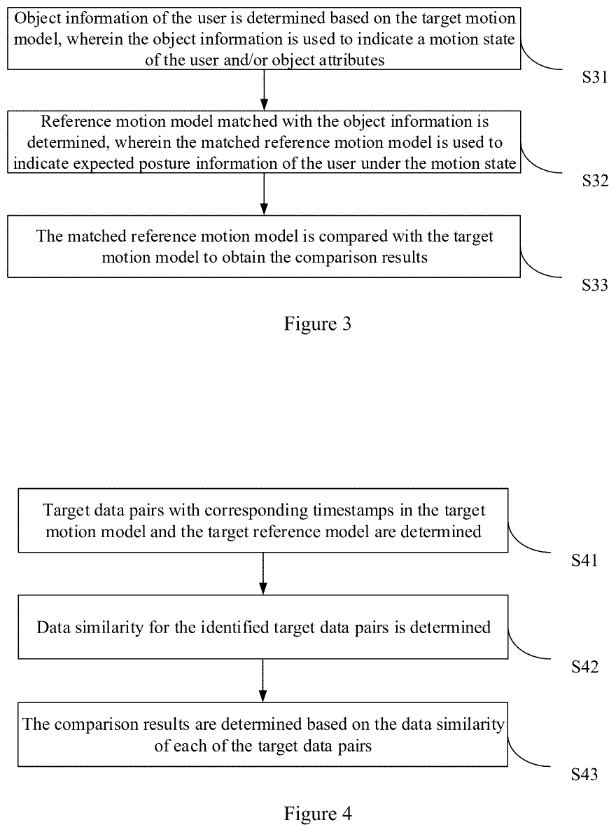

In an optional embodiment, as shown in FIG. 3, when the target reference model is a reference motion model, the above step of comparing the target motion model with the target reference model to obtain the comparison results includes the following steps:
step S31, object information of the user is determined based on the target motion model, wherein the object information is used to indicate a motion state of the user and/or object attributes;
step S32, a reference motion model matched with the object information is determined, wherein the matched reference motion model is used to indicate expected posture information of the user under the motion state; and
step S33, the matched reference motion model is compared with the target motion model to obtain the comparison results.

In the embodiments of the present disclosure, the target reference model includes the reference motion model and medical image data. The reference motion model and medical image data are stored in a database in advance. The reference motion model is used to indicate the expected posture information corresponding to the user, and the medical image data is used to indicate the expected skeletal state corresponding to the user.

Object information includes the motion state of the user and/or object attributes. The motion state includes, but is not limited to, motion types (e.g., specific fitness exercises, postures, cycling, and yoga moves), duration of activity, methods of movement (e.g., exercise with specific equipment), and location of the activity (e.g., indoors, and outdoors); object attributes include, but are not limited to, gender (e.g., male or female), age, health status (e.g., suffering from specific skeletal diseases), and other attributes related to the user.

Multiple target reference models can be set in advance, each target reference model can be set with a corresponding type label indicating a preset motion state and/or object attributes.

The user's object information can be compared with the tags of different reference motion models to find the reference motion model that best matches the object information of the user.

If it is determined that multiple reference motion models are matched with the object information, the priority of each piece of object information of the user can be determined. Specifically, the reference motion model that matches the object information with the highest priority is identified and selected. This selected reference motion model is then compared with the target motion model to obtain the comparison results.

Here, any one of pattern recognition algorithms, statistical analysis algorithms, optimization algorithms, machine learning algorithms, or artificial intelligence algorithms can be used to compare the target motion model with the reference motion model to obtain the comparison results.

Through this processing method, the target motion model and the reference motion model can be more accurately analyzed and compared, thereby obtaining more accurate comparison results.

In the embodiments of the present disclosure, the reference motion model can be a motion model determined based on the motion monitoring data of the user to indicate the expected posture information of the user. Additionally, the reference motion model can also be a motion model determined based on the motion monitoring data of other objects to indicate the expected posture information of the user, wherein the other objects can be objects related to the user, for example, object information of other objects is the same or highly similar to that of the user.

Through the above processing method, more suitable reference motion models can be identified for the target motion model, thereby obtaining more accurate comparison results and providing more accurate health intervention and correction information to the user.

In an optional embodiment, as shown in FIG. 4, the above step of comparing the target motion model with the target reference model to obtain the comparison results includes:
step S41, target data pairs with corresponding timestamps in the target motion model and the target reference model are determined;
step S42, a data similarity for the identified target data pairs is determined; and
step S43, the comparison results are determined based on the data similarity of each of the target data pairs.

In the embodiments of the present disclosure, depending on whether wearable devices are available, different types of the target motion model and the target reference model are considered. Algorithms dedicated to each case can be used to compare the target motion model with the target reference model.

Case 1: Wearable devices are available.

In this case, the target motion model can be referred to as a record sequence, and the target reference model can be referred to as a reference sequence. The reference sequence contains the expected posture information for each detection moment (e.g., Euler angles in three-dimensional space). The record sequence reflects the motion monitoring data collected from wearable devices during the entire intervention period. Here, the target data pairs corresponding to the same timestamp in the record sequence and the reference sequence can be determined, and then, the data similarity of the target data pairs can be determined. By comparing the data similarity between the reference sequence and the record sequence, a performance score can be obtained (e.g., a performance score between 0 and 100 can be obtained).

For example, the following reference sequence and record sequence each contain data for 5 timestamps, where the Euler angles on the X, Y, Z axes are measured at each timestamp.

Reference sequence: <(35, 24, 67), (16, 47, 24), (−24, 95, −3), (−61, 35, 24), (56, 33, 5)>.

Record sequence: <(30, 42, 70), (12, 60, 2), (−24, 95, −3), (−61, 35, 24), (56, 33, 5)>.

At this point, the target data pairs (Euler angles) corresponding to the same timestamp in the reference sequence and the record sequence can be determined, and then, the data similarity of the target data pairs can be determined to obtain the data similarity for each target data pair. Finally, based on the data similarity of all target data pairs, the comparison results can be determined.

For instance, an average value of the data similarity of all target data pairs can be calculated and the average value is used as the comparison result.

Case 2: Wearable devices are not available.

In this case, the record sequence can be obtained from image sequences captured by camera devices, for example, from smartphone cameras or network cameras. A convolutional neural network model can be used to extract measurements from each video frame of the reference sequence and the record sequence. The convolutional neural network consists of two parts.

Convolutional layer: a stack of convolutional layers and max pooling layers used to interpret prominent features of the input video frames; the output of the convolutional layer is a flattened one-dimensional feature vector.

Fully connected layer: a set of dense layers used to calculate measurements based on the feature vectors extracted from the two input images; the measurements typically involve Euler angles to determine the posture change between the two input images.

For example, given an image represented as X0 and a video frame represented as X1, the convolutional layer is applied to X0 and X1, and corresponding posture feature vectors U0 and U1 are extracted. The fully connected layer combines the two feature vectors to output a vector Y reflecting the posture change from X0 to X1. The vector Y is added to the record sequence as a measurement value for a specific timestamp.

After obtaining the record sequence, the similarity between the record sequence and the reference sequence is measured using a dynamic time warping algorithm. For example, a cosine similarity metric can be used to calculate the similarity between the two record sequences and the reference sequence.

Finally, a similarity score(S) is converted into a performance score (P), which ranges from 0 to 100. Let L and H be predefined parameters reflecting the expected range of similarity scores, meaning the user is most likely to achieve similarity scores within the range of [L, H], then the performance score can be calculated as follows:

P=min{max{(S−L)/(H−L)*100,0},100}.

The performance score P is guaranteed to be between 0 and 100, and the choice of L and H determines the difficulty for the user to obtain a high performance score.

Through the above processing method, more accurate comparison results can be obtained, thereby providing users with more accurate health interventions.

In an optional embodiment, when the target reference model is medical image data, the above step of comparing the target motion model with the target reference model to obtain comparison results includes the following steps:
step S51, state information of monitoring parts of the user is extracted based on the target motion model, wherein the monitoring parts comprise but are not limited to the following parts: skeletons, joints and muscular systems; and
step S52, the state information of the target monitoring parts is compared with an expected skeletal state of the target monitoring parts in the medical image data to obtain the comparison results.

As described above, the target reference model includes the reference motion model and medical image data, where the reference motion model and medical image data are stored in a database in advance. The medical image data is used to indicate the expected skeletal state corresponding to the user, which specifically includes MRI, CT, X-ray, and other image data.

For example, the medical image data can be models indicating the expected skeletal state, such as spinal alignment models, joint deformity models, cervical lordosis models, and cervical kyphosis models.

At this point, the skeletal state information of the target skeletal part of the user can be extracted based on the target motion model, for example, the spinal state information, joint state information, cervical spine state information, etc.

The skeletal state information can then be compared with the expected skeletal state in the medical image data to obtain the comparison results. Through these comparison results, the health status information of the target skeletal part of the user such as existing lesions or potential lesions can be determined.

In the embodiments of the present disclosure, the skeletal state of the target skeletal part of the user can be continuously monitored and assessed by monitoring the motion of the user in real-time, thereby allowing for longitudinal tracking of individual spinal and joint health and facilitating long-term evaluation of intervention effects.

In an optional embodiment, based on the comparison results, to the above steps of generating health intervention and correction information based on the comparison results, and sending the health intervention and correction information to the health intervention device include the following steps:
step S61, a model difference between the target motion model and the target reference model is determined based on the comparison results;
step S62, a health intervention strategy matched with the model difference is determined;
step S63, a health intervention device matched with the health intervention strategy is determined and the health intervention and correction information matched with the health intervention strategy is generated; and
step S64, the health intervention and correction information is sent to the matched health intervention device.

In the embodiments of the present disclosure, algorithms enabled by artificial intelligence can be used to identify deviations of the target motion model from standard norms (i.e., model differences) and appropriate intervention measures can be recommended to the users based on these deviations.

The comparison results can be input into an artificial intelligence algorithm for analysis to obtain the model differences, where these model differences are used to indicate deviations of data in the target motion model from standard values. Subsequently, health intervention strategies matching these model differences can be searched for in an associated table; these health intervention strategies include, but are not limited to, video guidance, tactile feedback from wearable devices, active intervention using devices like resistance bands, and therapeutic intervention using robots to aid in recovery.

After determining the health intervention strategies, health intervention devices compatible with these strategies can be determined, for example, the health intervention devices can be devices within sensors or other devices such as massage chairs. Then, health intervention and correction information compatible with the health intervention devices can be generated.

Health intervention and correction information is used to indicate intervention measures for health intervention on the users. These intervention measures include active intervention measures and passive intervention measures.

For active intervention measures, the health intervention and correction information can be intervention command information. By sending this intervention command information to health intervention devices, specific operations can be controlled, such as operations related to tactile feedback and elasticity adjustment. Through this processing method, the users can be actively guided to perform specific rehabilitation movements.

For passive intervention measures, the health intervention and correction information can be voice guidance information or video guidance information. For example, the voice guidance information can be audio messages guiding the users to perform specific rehabilitation movements; video guidance information can be videos guiding the users to perform specific rehabilitation movements.

In the above embodiments, target motion models are aligned with target reference models through algorithms supported by artificial intelligence, and the users are guided through correct movements via visual detection or wearable device detection, thereby providing automated therapy. Here, motion states of the users can also be automatically adjusted based on real-time feedback from artificial intelligence, allowing for physical therapy and real-time feedback to be delivered to the users without human intervention.

In an optional embodiment, generating health intervention and correction information based on the comparison results includes:
generating health intervention and correction information matched with the health intervention device based on the comparison results, where the health intervention and correction information includes types such as vibration information, video information, audio information, text information, and command information.

The health intervention device can be sensors that collect motion monitoring data or other devices capable of communicating with the target application (or electronic device). For example, the health intervention device can be a smart massager, or a smart robot.

At this point, health intervention and correction information compatible with the health intervention device can be generated; for example, if the health intervention device is a player, video-type health intervention and correction information or audio-type health intervention and correction information can be generated. If the health intervention device is a massage chair, then command-type health intervention and correction information can be generated, which can control the operation of the massage chair.

In an optional embodiment, the above step of acquiring various motion monitoring data obtained by detecting a user in a target state includes the following step:
acquiring various body state data obtained by performing posture measurement on the user in a sleep state through sensors of various dimensions, wherein the body state data includes at least one of the following types of data: sleep posture monitoring data, body photoelectric monitoring signals, body myoelectric monitoring signals and body vital sign signals.

In the embodiments of the present disclosure, sleep posture monitoring data is used to indicate posture data of the body of the user in a sleep state, body electromyography signals are used to indicate the signals obtained from electromyography of the body in the sleep state, and body vital signs signals are used to indicate the signals obtained from monitoring vital signs of the body in the sleep state.

Sensors in various dimensions can be used to measure the posture of the user, thereby obtaining various body state data. For example, a posture detector preset near the body of the user can be used to detect the posture data of the body of the user in the sleep state, such as placing the posture detector around the user's cervical spine, shoulders, lumbar spine, ankles, etc. For example, by placing the posture detector around the cervical spine of the user, the cervical spine posture of the user can be detected. Similarly, photoplethysmography sensors preset near the body of the user can be used for photoplethysmography detection to obtain body photoplethysmography signals. Likewise, electromyography sensors preset on the body of the user can be used for electromyography detection to obtain body electromyography signals. Similarly, vital signs sensors preset on the body of the user can be used for vital signs detection to obtain vital signs signals.

Additionally, image sensors can be used to collect images of the user in a sleep state, and through analysis of collected images, the sleep posture monitoring data of the user can be determined. Alternatively, sound collectors can be used to collect sounds made by the user in the sleep state, such as snoring sounds, breathing sounds, to predict the sleep posture monitoring data of the user.

After obtaining the body state data, the target motion model of the user can be determined based on the body state data; at this point, the target motion model can be understood as a sleep posture model determined based on the sleep posture monitoring data obtained from the user in the sleep state. In the case where the target reference model is a sleep reference model, the above step of comparing the target motion model with the target reference model to obtain the comparison results includes the following step:
comparing the sleep posture model with the sleep reference model to obtain the comparison results, wherein the sleep reference model is used to indicate an expected sleep posture of the user.

In the case of a target state being a sleep state, posture detection can also be performed on the users in the sleep state to obtain sleep posture monitoring data. Afterwards, based on this sleep posture monitoring data, a sleep posture model can be determined. Through this sleep posture model, state information of the user at sleep moments can be indicated, such as sleep posture information.

Next, the sleep posture model can be compared with the target reference model to obtain the comparison results, and based on these comparison results, health intervention and correction information can be generated and sent to the health intervention device.

For example, a cervical spine posture of a user in a sleep state can be detected to obtain sleep posture monitoring data. Based on this sleep posture monitoring data, a sleep posture model is determined, and this sleep posture model is compared with a target reference model indicating a standard sleeping posture of the cervical spine posture to obtain comparison results. For instance, the sleep posture monitoring data can be compared with medical image data under the standard sleeping posture to obtain the comparison results.

Through these comparison results, it can be determined whether the cervical spine of the user is in the expected sleep posture. When the user is not in the expected sleep posture, health intervention and correction information can be generated and sent to the health intervention device.

In an optional embodiment, the above steps of generating health intervention and correction information based on the comparison results, and sending the health intervention and correction information to the health intervention device include:
in response to determining that a posture difference between the sleep posture of the user and the expected sleep posture is large based on the comparison results, sending the health intervention and correction information to a sleep intervention device of the user, wherein the health intervention and correction information is used to adjust sleep posture of the user through the sleep intervention device. Here, the posture of the user can be intervened through automatic inflation or mechanical adjustment, and through the above intervention method, areas such as pressure sores can be automatically decompressed.

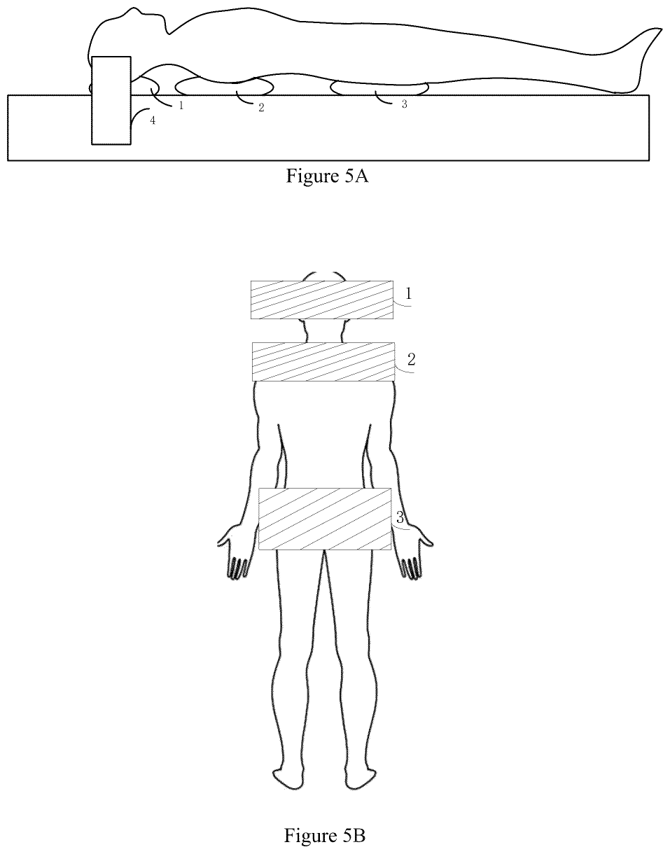

The health intervention device can be a sleep intervention device; or can be other auxiliary devices. The sleep intervention device can be an automatically inflatable pad or a mechanical pad that has a built-in pressure sensor. The sleep intervention device can also contain sub-devices, where each sub-device can be a pad or a mechanical pad that contain multiple inflation zones. For example, as shown in FIGS. 5A and 5B, a pad or a mechanical pad 1 can be set under a head area, a pad or a mechanical pad 2 can be set in an area extending from a scapula to below a neck, and another pad or a mechanical pad 3 can be set in an area from buttocks to a waist. By setting the sleep intervention device as shown in FIGS. 5A and 5B, compared to daytime, the sleep time at night is more sufficient, so it is possible to improve the problem of anterior spinal inclination during sleep. By setting the pad or mechanical pad 1, the pad or mechanical pad 2, and the pad or mechanical pad 3, it is possible to lift shoulders and waist of the body, thus mitigating the problem of anterior spinal inclination.

In the embodiments of the present disclosure, the inflation amount of the pad or mechanical pad can be set through inflation. For example, based on the body state data detected by sensors, the inflation state of the pad or mechanical pad can be adjusted. For instance, assuming that the sleep intervention device contains multiple pads, and each pad contains multiple inflation zones, then the following parameters about the pads can be determined based on the body state data detected by sensors: the pads that need to be adjusted, the inflation zone within the adjusted pads, and the inflation parameters for the inflation zone such as inflation volume, deflation volume, inflation time, etc.

For example, based on the electromyographic signals collected while the user is in a sleep state, a muscle tension level of the user during sleep can be determined. Based on this muscle tension level, the inflation parameters for the inflation zone of the pad to be adjusted can then be determined. By adjusting the inflation zone according to these inflation parameters, a muscle state of the user can be relaxed.

In the embodiments of the present disclosure, a sleep intervention device can also be customized for each individual based on medical diagnoses and recommendations (e.g., photos, X-rays) through 3D printing. This way, it is possible to achieve personalized customization of the sleep intervention device for the users, thereby generating a sleep intervention device that matches a body condition of each user more closely. Here, the sleep intervention device printed through 3D printing can be a mechanical pad that matches a spinal health state of the user.

In the embodiments of the present disclosure, the placement of the pad or mechanical pad is not limited to the positions shown in FIGS. 5A and 5B, but can also be set in other areas, not specifically limited by the present disclosure, as long as it can be implemented.

In the embodiments of the present disclosure, as shown in FIG. 5A, a terminal device 4, such as a smartphone, can also be placed on the side of the body of the user, where the terminal device must be able to fully capture the body of the user. The placement position of the terminal device is not specifically limited, as long as it can capture the entire body of the user. Through this terminal device, the user's visual imaging, breathing sounds, and other information can be collected. Through these visual imaging and/or breathing sounds, the body state data of the user in the sleep state can be determined. Through this body state data, the inflation state of the pad or mechanical pad can be adjusted. Alternatively, the terminal device can also use microwave detection to detect the body state data of the user in the sleep state.

In specific embodiments, the pad or mechanical pad 1 in the head area can be lowered. After lowering the pad or mechanical pad 1, if the body posture of the user does not meet preset requirements, the pad or mechanical pad 2 can be inflated. By inflating the pad or mechanical pad 2, the head area can be lowered. Additionally, the pad or mechanical pad 2 in the scapular area can also be lowered. After lowering the pad or mechanical pad 2, if the body posture of the user still does not meet the preset requirements, the pad or mechanical pad 3 can be inflated. By inflating pad or mechanical pad 3, the direction from the scapular area to the waist area can provide correction for lumbar anterior inclination.

Additionally, the sleep intervention device can be a smart massage pillow; for example, health intervention and correction information can be sent to this smart massage pillow, and the smart massage pillow can adjust its state, thereby adjusting the cervical spine posture of the user, and thus achieving the cervical spine of the user in the expected sleep posture, improving the sleep quality of the user.

In the embodiments of the present disclosure, the type and placement of the sleep intervention device can be set according to the specific problems requiring improvement including ones that are not specified by the present disclosure.

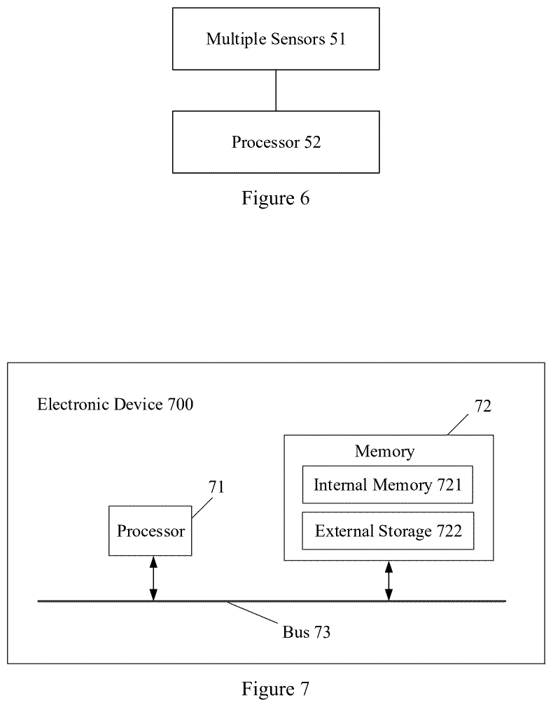

Referring to FIG. 6, FIG. 6 illustrates a schematic diagram of a health intervention and correction system based on vision and sensors provided by the embodiments of the present disclosure. The system includes multiple sensors 51 and a processor 52.

The multiple sensors 51 are configured to perform motion detection on a user in a target state to obtain various motion monitoring data.

The processor 52 is configured to acquire the various motion monitoring data and determine a target motion model of the user based on the various motion monitoring data, wherein the target motion model is used to indicate state information of the user at each detection moment; compare the target motion model with a target reference model to obtain comparison results; and generate health intervention and correction information based on the comparison results, and send the health intervention and correction information to a health intervention device.

The various motion monitoring data can be motion monitoring data collected by various sensors detecting the movement of the user. The data types of the various motion monitoring data are not completely identical.

The various sensors include wearable devices and camera devices.

Here, the motion monitoring data is obtained by performing motion detection on various monitoring parts of the user, where the monitoring parts include but are not limited to the following parts: skeletons, joints, and muscles; data types of the various motion monitoring data include but are not limited to the following types: visual images, photoelectric data, neuroelectrophysiological data, audio data, and speed data.

Through the motion monitoring data, the following related motion data of the user can be determined: a motion posture of the user, a motion duration, a motion speed, a motion mode (for example, cycling or walking), a body state during a motion (such as a heart rate, a blood oxygen concentration, a blood pressure, and a pulse), a motion trajectory, and other data associated with the motion.

In the embodiments of the present disclosure, a target application can be installed in the electronic device. Here, the processor in the electronic device can obtain the motion monitoring data collected by the various sensors and send this motion monitoring data to the target application. After obtaining the motion monitoring data, the target application can perform the following steps S103 to S107. Here, if the sensor is a wearable device, a client that communicates and connects with the wearable device can be installed in the electronic device. The client can obtain the motion monitoring data collected by the wearable device through the communication connection and send this motion monitoring data to the target application.

Additionally, communication connections between the processor with the various sensors can be established, for example, a communication channel between the processor and wearable devices. Afterward, the wearable devices can transmit motion monitoring data to the processor through this communication channel, and upon receiving this motion monitoring data, the above steps S103 to S107 can be executed.

The multiple sensors include wearable devices and camera devices, where the wearable devices and the camera devices are configured to connect to the processor via wireless or wired connections.

After obtaining various motion monitoring data, these various motion monitoring data can be fused together to obtain the target motion model.

The number of the target motion model can be multiple, and the data types included in different target motion models are different. That is, multiple different target motion models in the same time period can be generated using various types of motion monitoring data.

The target motion model can be represented by one or more of the following methods: a mathematical model, a statistical model, a biomechanical model, a kinematic model, or a machine learning model.

The target motion model can be transformed into a motion sequence; wherein the motion sequence can indicate the state information of the user at each detection moment. Here, the detection moment is a moment when the sensor performs motion detection on the user.

The target reference model can be a reference model generated through normal motion monitoring data of the user, or a reference model generated through clinical standards. The expected posture information of the subject can be determined under normal circumstances through the target reference model.

The target reference model can be represented by one or more of the following methods: a mathematical model, a statistical model, a biomechanical model, a kinematic model, or a machine learning model.

In the embodiments of the present disclosure, first, various motion monitoring data obtained by detecting the user are acquired; then, the target motion model of the user is determined based on the various motion monitoring data, state information of the user at each detection moment can be determined through the target motion model; subsequently, health intervention and correction information of the user can be generated by comparing the target motion model with a target reference model, and the health intervention and correction information is sent to the health intervention device.

In the above embodiments, by determining the target motion model based on the various motion monitoring data of the user, a personalized health model can be generated for the user; by comparing the target motion model with the target reference model to generate health intervention and correction information, more accurate health intervention and correction information can be generated for the user, thereby providing more precise intervention measures for the user.

In an optional embodiment, the processor 52 includes a processing module, a comparison module, and an intervention module.

The processing module is configured to obtain the various motion monitoring data and, based on these data, determine the target motion model of the user.

The processing module can determine the target motion model of the user in the manner described in the above examples, which is not detailed here again.

The comparison module is configured to compare the target motion model with the target reference model to obtain the comparison results.

The comparison module compares the target motion model with the target reference model in the manner that has been described in the above examples.

The intervention module is configured to generate health intervention and correction information based on the comparison results and send this health intervention and correction information to the health intervention device.

Here, the intervention model can generate health intervention and correction information in the manner that has been described in the above examples.

A health intervention and correction system based on vision and sensors will be introduced with specific embodiments. This health intervention and correction system based on vision and sensors, with user authorization, can monitor and intervene in the bending movements and wear of the head spine and joints, as well as related neural and cardiopulmonary functions.

The health intervention and correction system based on vision and sensors includes sensors and a processor, where the sensors include wearable devices, camera equipment, and monitoring modules of the wearable devices.

The wearable devices can be divided into different monitoring modules for detection, including a module 1, a module 2, a module 3, and a module 4. The module 1 can be used for detecting ear hooks, smart glasses, smart headbands. The module 2 can be used for detecting smart chest straps. The module 3 can be used for detecting smart waist belts. The module 4 can be used for detecting visual sensors. Through these monitoring modules, various motion monitoring data obtained from detection of the user in a target state can be collected.

For example, the module 1 can be used to detect changes in the cervical spine angle of ear hooks, glasses, and headbands. The module 2, through a chest strap, can perform electrocardiogram heart rate and breathing detection and assist head sensors in locating thoracic and lumbar spine angles. The module 3, through a waist belt or limb joint rings, can detect wear of the spine and limb joints and record electromyographic signals of large muscles near the spinal joints, with large limb joints being the knee, ankle, elbow, or wrist. The module 4 can collect visual images through visual sensors.

Each monitoring module is equipped with a Bluetooth chip, allowing it to establish a communication connection with the client in the electronic device.

For instance, the module 1 includes a control chip, Bluetooth chip, battery, nine-axis and vibration sensors, vibration motor. It may further include a dual-color LED indicator light, buzzer. The module 2 contains a control chip, Bluetooth chip, battery, nine-axis and vibration sensors, electrocardiogram monitoring module. The module 3 contains a control chip, Bluetooth chip, battery, electromyography and muscle motion monitoring module (EMG&MMG), vibration and audio collection chip, and directional microphone. It may further include a dual-color LED indicator light, etc.

Here, the headband, using the module 1, is placed on the head. The chest strap, using the module 2, is placed on the left chest area. The waist belt, using the module 3, is placed on the lower back, each having additional functions to record heart rate, electrocardiogram, breathing status, lung capacity, breathing rate, oxygen saturation, bowel sounds, and abdominal pressure detection as well as to detect and intervene in the tension and movement of the lumbar spine. Depending on the situation, the users may use one, two, or all three modules individually.

In the embodiments of the present disclosure, motion monitoring data collected by the respective wearable devices through the modules 1 to 4 can be transmitted to the client through the Bluetooth chip for processing. Here, the client can determine the target motion model of the user based on the various motion monitoring data, where the target motion model is used to indicate the state information of the user at each detection moment. The client compares the target motion model with the target reference model to obtain the comparison results, generates health intervention and correction information based on the comparison results, and sends this health intervention and correction information to the health intervention device.

Here, the specific embodiment of the client has been described above and will not be mentioned here again.

To clarify, in the above-described specific embodiments, the order of writing each step does not imply a strict execution order, and the specific execution order should be determined based on its function and possible internal logic.

The description of the processing flow of each module in the device and the interaction process between the modules has been explained in the above examples and will not be mentioned here again.

Regarding the health intervention and correction method based on vision and sensors depicted in FIG. 1, the embodiment of the present disclosure further provides an electronic device 700, as shown in FIG. 7, an illustrative diagram of the electronic device 700 structure provided by the embodiment of the present disclosure, including:
a processor 71, a memory 72, and a bus 73. The memory 72 is used to store execution instructions, including an internal memory 721 and an external storage 722. The internal memory 721, also called internal storage, is used to temporarily store operational data in the processor 71 and exchange data with external storage such as hard drives 722. The processor 71 exchanges data with the external storage 722 through the internal memory 721. When the electronic device 700 operates, the processor 71 communicates with the memory 72 through the bus 73, enabling the processor 71 to execute the following instructions:
acquiring various motion monitoring data obtained by detecting a user in a target state;
determining a target motion model of the user based on the various motion monitoring data, wherein the target motion model is used to indicate state information of the user at each detection moment;
comparing the target motion model with a target reference model to obtain comparison results; and
generating health intervention and correction information based on the comparison results, and sending the health intervention and correction information to a health intervention device.

The embodiment of the present disclosure also provides a computer-readable storage medium, which stores a computer program, when executed by a processor, performs the steps of the health intervention and correction method based on vision and sensors as described in the above method examples. The storage medium can be a volatile or non-volatile computer-readable storage medium.

The embodiment of the present disclosure also provides a computer program product, which carries program code, the instructions included in the program code are for executing the steps of the health intervention and correction method based on vision and sensors as described in the above method examples, specifics can refer to the above method examples, not detailed here again.

The above computer program product can be specifically implemented through hardware, software, or a combination thereof. In an optional embodiment, the computer program product is specifically embodied as a computer storage medium, in another optional embodiment, the computer program product is specifically embodied as a software product, such as a Software Development Kit (SDK), etc.

Those skilled in the field can clearly understand that, for convenience and brevity of description, the specific working processes of the systems and devices described above can refer to the corresponding processes in the above examples, not detailed here again. In several embodiments provided by this publication, the disclosed systems and methods can be implemented in other ways. The device embodiments described above are merely illustrative, for example, the division of the units is just a logical functional division, in actual implementation, there can be other division methods; for example, multiple units or components can be combined or integrated into another system, or some features can be ignored, or not executed. Moreover, the coupling or direct coupling or communication connection shown or discussed between each other can be through some communication interfaces, devices or units' indirect coupling or communication connection, can be electrical, mechanical, or other forms.

The units described as separate components may or may not be physically separate, the components shown as units may or may not be physical units, that is, they can be located in one place, or distributed over several network units. Part or all of the units can be selected according to the actual needs to achieve the purpose of the embodiment of this scheme.

Additionally, in various embodiments of this publication, each functional unit can be integrated into one processing unit, or each unit can physically exist separately, or two or more units can be integrated into one unit.

The functionality if implemented in the form of software functional units and sold or used as separate products, can be stored in a processor-executable non-volatile computer-readable storage medium. Based on such understanding, the essence of the technical scheme of this publication or the part of the technical scheme that contributes to the existing technology can be embodied in the form of a software product, which is stored in a storage medium, including several instructions to enable an electronic device (which can be a personal computer, server, or network device, etc.) to execute all or part of the steps of the methods described in various embodiments of this publication. The storage medium includes various media that can store program codes such as a USB flash drive, mobile hard disk, read-only memory (ROM), random access memory (RAM), disk or CD, etc.

Finally, it is worth noting that the above examples are only to illustrate the technical solutions of this publication, not limiting, although this publication has been described in detail with reference to the foregoing examples. Those who are familiar with the field should understand: any person working in the field within the technical scope disclosed by this publication, can still modify or equivalently replace the technical solutions recorded in the foregoing examples, and these modifications, changes, or equivalent replacements do not make the essence of the corresponding technical solutions deviate from the spirit and scope of the technical solutions of the embodiments of this publication, which should be covered within the protection scope of this publication. Therefore, the protection scope of this publication should be determined by the appended claims.

1. A health intervention and correction method based on vision and sensors, comprising:
acquiring various motion monitoring data obtained by detecting a user in a target state;
determining a target motion model of the user based on the various motion monitoring data, wherein the target motion model is used to indicate state information of the user at each detection moment;
comparing the target motion model with a target reference model to obtain comparison results; and
generating health intervention and correction information based on the comparison results, and sending the health intervention and correction information to a health intervention device.

2. The method according to claim 1, wherein acquiring the various motion monitoring data obtained by detecting the user in the target state comprises:
acquiring motion monitoring data obtained by performing motion detection on the user by a plurality of wearable devices in communication connection with a terminal device to obtain the various motion monitoring data.

3. The method according to claim 2, wherein the various motion monitoring data is obtained by performing motion detection on various monitoring parts of the user, wherein the monitoring parts comprise but are not limited to the following parts: skeletons, joints and muscles; the motion monitoring data comprise but are not limited to the following types: visual images, photoelectric data, neuroelectrophysiological data, audio data and speed data.

4. The method according to claim 1, wherein determining the target motion model of the user based on the various motion monitoring data comprises:
transforming each of the various motion monitoring data into motion posture data of the user; and
performing data fusion on the motion posture data obtained after transforming the various motion monitoring data based on the detection moment of the motion posture data to obtain the target motion model.

5. The method according to claim 4, wherein performing data fusion on the motion posture data obtained after transforming the various motion monitoring data based on the detection moment of the motion posture data to obtain the target motion model comprises:
determining motion posture data with the same detection moment in the motion posture data obtained after transforming the various motion monitoring data;
processing the motion posture data with the same detection moment to obtain target motion posture data at the detection moment; and
determining the target motion model based on the target motion posture data at each detection moment.

6. The method according to claim 1, wherein the target reference model is a reference motion model, and comparing the target motion model with the target reference model to obtain the comparison results comprises:
determining object information of the user based on the target motion model, wherein the object information is used to indicate a motion state of the user and/or object attributes;
determining a reference motion model matched with the object information, wherein the matched reference motion model is used to indicate expected posture information of the user under the motion state; and
comparing the matched reference motion model with the target motion model to obtain the comparison results.

7. The method according to claim 1, wherein comparing the target motion model with the target reference model to obtain the comparison results comprises:
determining target data pairs with corresponding timestamps in the target motion model and the target reference model;
determining a data similarity of the target data pairs; and
determining the comparison results based on the data similarity of each of the target data pairs.

8. The method according to claim 1, wherein generating health intervention and correction information based on the comparison results, and sending the health intervention and correction information to the health intervention device comprises:
determining a model difference between the target motion model and the target reference model based on the comparison results;
determining a health intervention strategy matched with the model difference;
determining a health intervention device matched with the health intervention strategy and generating the health intervention and correction information matched with the health intervention strategy; and
sending the health intervention and correction information to the matched health intervention device.

9. The method according to claim 1, wherein the target reference model is medical image data, and comparing the target motion model with the target reference model to obtain comparison results comprises:
extracting state information of monitoring parts of the user based on the target motion model, wherein the monitoring parts comprise but are not limited to the following parts: skeletons, joints and muscular systems; and
comparing the state information of the target monitoring parts with an expected skeletal state of the target monitoring parts in the medical image data to obtain the comparison results.

10. The method according to claim 1, wherein the target motion model is a sleep posture model determined based on body state data obtained from the user in a sleep state, and the target reference model is a sleep reference model;
acquiring various motion monitoring data obtained by detecting a user in a target state comprises:
acquiring various body state data obtained by performing posture measurement on the user in a sleep state through sensors of various dimensions, wherein the body state data comprises at least one of the following types of data: sleep posture monitoring data, body photoelectric monitoring signals, body myoelectric monitoring signals and body vital sign signals; and
comparing the target motion model with the target reference model to obtain the comparison results comprises:
comparing the sleep posture model with the sleep reference model to obtain the comparison results, wherein the sleep reference model is used to indicate an expected sleep posture of the user.

11. The method according to claim 10, wherein generating health intervention and correction information based on the comparison results, and sending the health intervention and correction information to the health intervention device comprises:
in response to determining that a posture difference between the sleep posture of the user and the expected sleep posture is large based on the comparison results, sending the health intervention and correction information to a sleep intervention device of the user, wherein the health intervention and correction information is used to adjust sleep posture of the user through the sleep intervention device.

12. The method according to claim 1, wherein generating health intervention and correction information based on the comparison results comprises:
generating health intervention and correction information matched with the health intervention device based on the comparison results, wherein the health intervention and correction information comprises the following types: vibration information, video information, audio information, textual information and command information.

13. A health intervention and correction system based on vision and sensors, comprising multiple sensors and a processor, wherein
the multiple sensors are configured to perform motion detection on a user in a target state to obtain various motion monitoring data; and
the processor is configured to acquire the various motion monitoring data and determine a target motion model of the user based on the various motion monitoring data, wherein the target motion model is used to indicate state information of the user at each detection moment; compare the target motion model with a target reference model to obtain comparison results; and generate health intervention and correction information based on the comparison results, and send the health intervention and correction information to a health intervention device.

14. The system according to claim 13, wherein the processor comprises a processing module, a comparison module, and an intervention module;
the processing module is configured to acquire the various motion monitoring data and determine the target motion model of the user based on the various motion monitoring data;
the comparison module is configured to compare the target motion model with the target reference model to obtain the comparison results; and
the intervention module is configured to generate health intervention and correction information based on the comparison results and send the health intervention and correction information to the health intervention device.

15. The system according to claim 13, wherein the multiple sensors comprise wearable devices and camera devices, wherein the wearable devices and the camera devices are configured to connect to the processor via wireless or wired connections.

16. An electronic device, comprising: a processor, a memory, and a bus; wherein the memory stores machine-readable instructions executable by the processor; when the electronic device is operational, the processor communicates with the memory via the bus, and when the machine-readable instructions are executed by the processor, they perform the steps of the health intervention and correction method based on vision and sensors according to claim 1.

17. A computer-readable storage medium storing a computer program, wherein the computer program, when executed by a processor, causing the processor to perform the steps of the health intervention and correction method based on vision and sensors according to claim 1.
